(19) United States
(12) Patent Application Publication
(10) Pub. No.: US 2016/0262712 A1
(43) Pub. Date: Sep. 15, 2016

(54) X-RAY PARADOXICAL PULSE BI-PLANAR SYNCHRONOUS REAL-TIME IMAGING DEVICE AND IMAGING METHOD

(71) Applicant: Beijing East Whale Image Technology Co., Ltd., Beijing (CN)

(72) Inventors: Xun ZHU, Yizhuang, Beijing (CN); Xingbai HE, Yizhuang, Beijing (CN); Jun ZHANG, Yizhuang, Beijing (CN)

(21) Appl. No.: 15/031,420

(22) PCT Filed: Jan. 17, 2014
(86) PCT No.: PCT/CN2014/000057

§ 371 Date: Apr. 22, 2016

(51) Int. Cl. A61B 6/00

(52) U.S. Cl. CPC A61B 6/5205; A61B 6/4441; A61B 6/464; A61B 6/4266; A61B 6/4014

(57) ABSTRACT

An X-ray paradoxical pulse bi-planar synchronous real-time imaging device and imaging method are disclosed wherein the device includes the second C-arm and the first C-arm slidably set on the said second C-arm, wherein the first X-ray generator and the first image receiver are mounted on the said first C-arm, and the second X-ray generator and the second image receiver are mounted on the said second C-arm. An imaging control device controls the pulse cycle and width of the first X-ray pulse and second X-ray pulse, so that the first X-ray pulse and second X-ray pulse do not mutually interfere or have low degree of interference in one cycle. Therefore, the synchronous real-time X-ray imaging of the first imaging device and second imaging device can be realized. Furthermore, the imaging control device can display the X-ray images in real time and synchronously by processing the X-ray images.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The invention relates to an X-ray imaging device and imaging method, especially relating to an X-ray paradoxical pulse bi-planar synchronous real-time imaging device and imaging method.

2. Description of Related Art Including Information Disclosed Under 37 CFR 1.97 and 37 CFR 1.98.

When conducting the operations such as bone fixation operation, the surgeons often need to observe the precise location of metallic implants or mobile surgical instruments for more precise positioning.

The United States Patent U.S. Pat. No. 4884293 describes a bi-planar imaging method, which is adopted with two sets of completely independent imaging systems or two C-arms. One set of the imaging system is fixed on the ground of the operating room and another set of system is suspended from the guide rail set at the ceiling. Although the two sets of imaging systems are installed cooperated with each other, the weakness of this method is that the imaging exposure of the two sets of imaging systems is respectively independent and can't be synchronous.

European Patent EP0917856A1 describes another bi-planar imaging method, by which two sets of imaging systems image on two displays one by one after sequential exposure. The method solves the problems of interference and scattering of two-way X-rays, but it can't display the real-time images of two angles synchronously.

BRIEF SUMMARY OF THE INVENTION

The first purpose of the invention is to provide an X-ray paradoxical pulse bi-planar synchronous real-time imaging device that can display the X-ray image in real time and synchronously.

The second purpose of the invention is to provide an X-ray paradoxical pulse bi-planar synchronous real-time imaging method that can display the X-ray image in real time and synchronously.

The technical proposal adopted in the invention to solve the first technical problem is as follows: a kind of X-ray paradoxical pulse bi-planar synchronous real-time imaging device, including the first C-arm, the second C-arm, support device, the first X-ray generator, the first image receiver, the second X-ray generator, the second image receiver and imaging control device;

The said first C-arm is slidably set on the said second C-arm; the said second C-arm is slidably set on the said support device;

The said first X-ray generator and the first image receiver are fixed on two axial ends of the said first C-arm respectively;

The said second X-ray generator and the second image receiver are fixed on two axial ends of the said second C-arm respectively;

Taking the intersecting line between the x-ray emission plane of the said first X-ray generator and the x-ray emission plane of the said second X-ray generator as the rotation axis, the said first X-ray generator and the first image receiver rotate along the rotation axis driven by the said first C-arm and the said second X-ray generator and the second image receiver also rotate along the rotation axis driven by the said second C-arm;

The said imaging control device includes: console, image processor, X-ray controller, the first high-voltage generator, the second high-voltage generator, network card, the first camera, the second camera, the first display, the second display and operation display;

The said image processor and X-ray controller are connected to the signal of the said console;

The said first high-voltage generator and second high-voltage generator are connected to the signal of the said X-ray controller and also connected to the first X-ray generator and the second X-ray generator respectively;

The said first camera and second camera are respectively connected to the said image processor and X-ray controller by the signal of the said network card;

The said first display, second display and operation display are connected to the signal of the said image processor respectively.

Alternatively, the said first X-ray generator is the first X-ray bulb tube and the said second X-ray generator is the second X-ray bulb tube.

Alternatively, the said X-ray paradoxical pulse bi-planar synchronous real-time imaging device also includes the first motor set on the said second C-arm, which is connected to the said console by the motor control signal.

Alternatively, the said X-ray paradoxical pulse bi-planar synchronous real-time imaging device also includes a foot switch, which is connected to the said console by signal.

The technical proposal adopted in the invention to solve the second technical problem is as follows: a method to realize the X-ray paradoxical pulse bi-planar synchronous real-time imaging by using the said X-ray paradoxical pulse bi-planar synchronous real-time imaging device, including the following steps:

S10. Control the motion of the said first motor to make the included angle between the directions of the X-ray generated from the first X-ray generator and the X-ray generated from the second X-ray generator a preset value;

S20. Select the operating mode of the said X-ray paradoxical pulse bi-planar synchronous real-time imaging device;

S30. The said first high-voltage generator and the second high-voltage generator are controlled by the said X-ray controller to generate the pulse high voltage. The said pulse high voltages generated by the said first high-voltage generator and the second high-voltage generator make the said first X-ray generator and second X-ray generator produce X-ray pulses, among which the X-ray pulse produced by the said first X-ray generator is the first X-ray pulse and the X-ray pulse produced by the said second X-ray generator is the second X-ray pulse. The phase of the said first X-ray pulse and that of the second X-ray pulse are different;

S40. The said first image receiver and second image receiver respectively receive the X-ray pulses generated when the first X-ray pulse and second X-ray pulse penetrate the detection object and generate the visible light signals;

S50. The said first camera and second camera respectively collect the visible light signals generated by the first image receiver and second image receiver and transfer to the said image processor by the network card;

S60. The said image processor displays the visible light signals collected by the said first camera on the first display and visible light signals collected by the said second camera on the second display.

Alternatively, in the said S20, the said operating modes include:

a. The first X-ray generator and the first image receiver operate, while the second X-ray generator and the second image receiver do not operate;

b. The second X-ray generator and the second image receiver operate, while the first X-ray generator and the first image receiver do not operate;

c. The first X-ray generator and the first image receiver as well as the second X-ray generator and the second image receiver operate simultaneously.

Alternatively, there is also S05 before the S10,

S05. Set the cycle top limit T of the said X-ray paradoxical pulse bi-planar synchronous real-time imaging device.

Alternatively, there is also S35 between the S30 and S40: the said first X-ray pulse's total cycle and the cycle top limit are the same, with the duration as T₁; the said second X-ray pulse's total cycle and the cycle top limit are the same, with the duration as T₂. The overlap between the said first X-ray pulse and the said second X-ray is Tₜ satisfying T₁+T₂≦T+Tt.

Alternatively, there is also S36 between the S30 and S40: the said first X-ray pulse's total cycle and the cycle top limit are the same, with the duration as T₁; the said second X-ray pulse's total cycle and the cycle top limit are the same, with the duration as T₂, satisfying T₁+T₂≦T.

Alternatively, in the said S35 or S36, the T₁ and T₂ are different.

The X-ray paradoxical pulse bi-planar synchronous real-time imaging device of the invention has the following beneficial effects: the said X-ray paradoxical pulse bi-planar synchronous real-time imaging device includes the second C-arm and the first C-arm slidably set on the said second C-arm. The first X-ray generator and the first image receiver are installed on both sides of the said first C-arm, constituting the first imaging device. The second X-ray generator and the second image receiver are installed on both sides of the said second C-arm, constituting the second imaging device. The said imaging control device can control the pulse cycle and width of the said first X-ray pulse and second X-ray pulse, so that the said first X-ray pulse and second X-ray pulse do not mutually interfere or have low interference degree in one cycle. Therefore, the synchronous real-time X-ray imaging of the said first imaging device and second imaging device can be realized. Further, the said imaging control device can display the X-ray images in real time and synchronously by processing the said X-ray images.

The X-ray paradoxical pulse bi-planar synchronous real-time imaging method of the invention has the following beneficial effects: the said first X-ray generator and second X-ray generator are controlled by the said X-ray controller to generate the first X-ray pulse and second X-ray pulse, among which the phases of the said first X-ray pulse and second X-ray pulse are different. Therefore, the said first X-ray pulse and second X-ray pulse do not mutually interfere or have low the interference degree in one cycle. Hence the synchronous real-time X-ray imaging of the said first imaging device and second imaging device can be realized. Further, the said imaging control device can display the X-ray images in real time and synchronously by processing the said X-ray images.

### BRIEF DESCRIPTION OF THE DRAWINGS

FIG. 1 is the structure diagram of the X-ray paradoxical pulse bi-planar synchronous real-time imaging device of the invention;

FIG. 2 is the structure diagram of the imaging control device of the invention;

FIG. 3 is a sequence diagram of the first X-ray pulse and second X-ray pulse of the invention;

FIG. 4 is another sequence diagram of the first X-ray pulse and second X-ray pulse of the invention;

The marks in the figures are as follows: 1—the first C-arm; 2—the second C-arm; 3—the first X-ray generator; 4—the first image receiver; 5—the second X-ray generator; 6—the second image receiver; 7—imaging control device; 71—console; 72—image processor; 73—X-ray controller; 74—the first high-voltage generator; 75—the second high-voltage generator; 76—network card; 77—the first camera; 78—the second camera; 79—the first display; 710—the second display; 711—operation display; 712—motor controller; 713—foot switch; 8—the first motor.

### DETAILED DESCRIPTION OF THE INVENTION

The technical proposals of the invention are further expounded with the combination of embodiments and attached figures.

### Embodiment 1

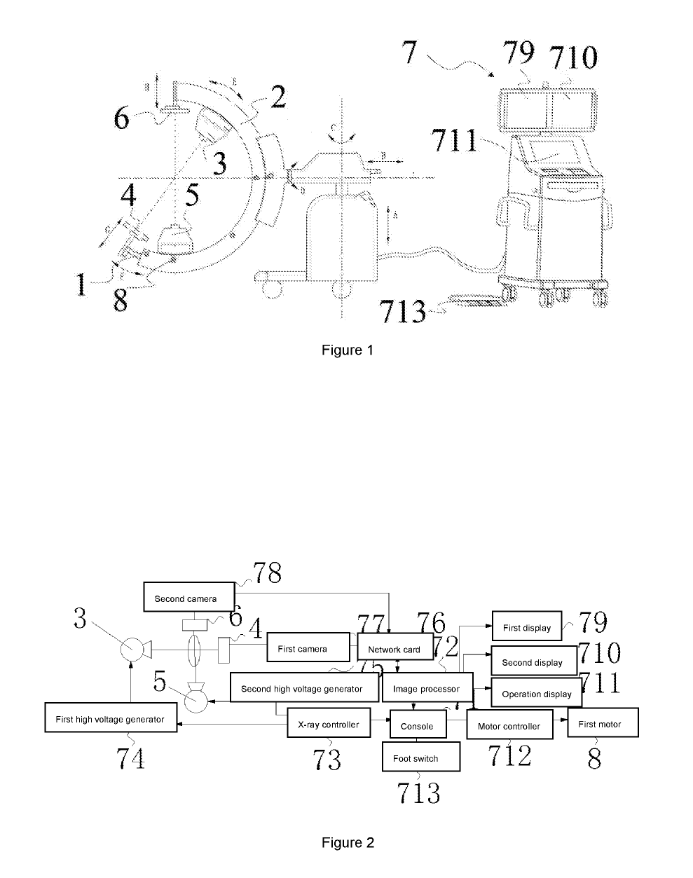

By reference to FIG. 1-4, this embodiment provides an X-ray paradoxical pulse bi-planar synchronous real-time imaging device, including the first C-arm 1, the second C-arm 2, support device, the first X-ray generator 3, the first image receiver 4, the second X-ray generator 5, the second image receiver 6 and imaging control device 7;

The said first C-arm 1 is slidably set on the said second C-arm 2 and the said second C-arm 2 is slidably set on said support device;

The said first X-ray generator 3 and the first image receiver 4 are respectively fixed on the two circumferential ends of the first C-arm 1;

The said second X-ray generator 5 and the second image receiver 6 are respectively fixed on the two circumferential ends of the first C-arm 2;

Taking the intersecting line between the x-ray emission plane of the said first X-ray generator 3 and the x-ray emission plane of the said second X-ray generator 5 as the rotation axis, the said first X-ray generator 3 and the first image receiver 4 rotate along the rotation axis driven by the said first C-arm 1 and the said second X-ray generator 5 and the second image receiver 6 also rotate along the rotation axis driven by the said second C-arm 2;

The said imaging control device 7 includes: console 71, image processor 72, X-ray controller 73, the first high-voltage generator 74, the second high-voltage generator 75, network card 76, the first camera 77, the second camera 78, the first display 79, the second display 710 and operation display 711;

The said image processor 72 and X-ray controller 73 are connected to the signal of the said console 71;

The said first high-voltage generator 74 and second high-voltage generator 75 are connected to the signal of the said X-ray controller 73 and connected to the first X-ray generator 3 and the second X-ray generator 5 respectively;

The said first camera 77 and second camera 78 are respectively connected to the said image processor 72 and X-ray controller 73 by the signal of the said network card 76;

The said first display 79, second display 710 and operation display 711 are connected to the signal of the said image processor 72 respectively.

The technical proposal adopted in the invention to solve the second technical problem is as follows: the said X-ray paradoxical pulse bi-planar synchronous real-time imaging device includes the second C-arm 2 and the first C-arm 1 slidably set on the said second C-arm 2. The first X-ray generator 3 and the first image receiver 4 are installed on both sides of the said first C-arm 1, constituting the first imaging device. The second X-ray generator 5 and the second image receiver 6 are installed on both sides of the said second C-arm 2, constituting the second imaging device. The said imaging control device can control the pulse cycle and width of the said first X-ray pulse and second X-ray pulse, so that the said first X-ray pulse and second X-ray pulse do not mutually interfere or have low interference degree in one cycle. Therefore, the synchronous real-time X-ray imaging of the said first imaging device and second imaging device can be realized. Further, the said imaging control device 7 can display the X-ray images in real time and synchronously by processing the said X-ray images.

In the embodiment, alternatively, the said first X-ray generator 3 is the first X-ray bulb tube and the said second X-ray generator 5 is the second X-ray bulb tube.

In the embodiment, alternatively, the said X-ray paradoxical pulse bi-planar synchronous real-time imaging device also includes the first motor 8 set on the said second C-arm 2. The said first motor 8 drives the said first C-arm to slide along the said second C-arm 2. The said first motor 8 is connected to the said console 71 by the signal of the motor controller 712 to control the motion of the said first motor 8 through the said console 71, hence to make different included angles of the said first imaging device and second imaging device.

In the embodiment, alternatively, the said X-ray paradoxical pulse bi-planar synchronous real-time imaging device also includes a foot switch 713, which is connected to the said console 71 by signal, so that the operating mode of the said X-ray paradoxical pulse bi-planar synchronous real-time imaging device can be selected through the foot switch 713. The said operating modes include: the first X-ray generator 3 and the first image receiver 4 operate; the second X-ray generator 5 and the second image receiver 6 operate; and the first X-ray generator 3 and the first image receiver 4 as well as the second X-ray generator 5 and the second image receiver 6 operate simultaneously.

### Embodiment 2

By reference to FIG. 1-4, this embodiment provides a method to realize the X-ray paradoxical pulse bi-planar synchronous real-time imaging by using the said X-ray paradoxical pulse bi-planar synchronous real-time imaging device, including the following steps:

S10. Control the motion of the said first motor 8 to make the included angle between the directions of the X-ray generated from the first X-ray generator 3 and the X-ray generated from the second X-ray generator 5 a preset value;

S20. Select the operating mode of the said X-ray paradoxical pulse bi-planar synchronous real-time imaging device;

S30. The said first high-voltage generator 74 and the second high-voltage generator 75 are controlled by the said X-ray controller 73 to generate the pulse high voltage. The said pulse high voltages generated by the said first high-voltage generator 74 and the second high-voltage generator 75 make the said first X-ray generator 3 and second X-ray generator 5 produce X-ray pulses, among which the X-ray pulse produced by the said first X-ray generator 3 is the first X-ray pulse and the X-ray pulse produced by the said second X-ray generator 5 is the second X-ray pulse. The phase of the said first X-ray pulse and that of the second X-ray pulse are different;

S40. The said first image receiver 4 and second image receiver 6 respectively receive the X-ray pulses generated when the first X-ray pulse and second X-ray pulse penetrate the detection object and generate the visible light signals;

S50. The said first camera 77 and second camera 78 respectively collect the visible light signals generated by the first image receiver 4 and second image receiver 7 and transfer to the said image processor 72 by the network card 76;

S60. The said image processor 72 displays the visible light signals collected by the said first camera 77 on the first display 79 and visible light signals collected by the said second camera 78 on the second display 710.

The X-ray paradoxical pulse bi-planar synchronous real-time imaging method of the invention has the following beneficial effects: the said first X-ray generator 3 and second X-ray generator 5 are controlled by the said X-ray controller 73 to generate the first X-ray pulse and second X-ray pulse, among which the phases of the said first X-ray pulse and second X-ray pulse are different. Therefore, the said first X-ray pulse and second X-ray pulse do not mutually interfere or have low the interference degree in one cycle. Hence the synchronous real-time X-ray imaging of the said first imaging device and second imaging device can be realized. Further, the said imaging control device can display the X-ray images in real time and synchronously by processing the said X-ray images.

In the embodiment, alternatively, in S10, the said preset value is 5-95° to observe the detection objects such as human body and/or object from multi-angles.

In the embodiment, alternatively, in S20, the said operating modes include: a. The first X-ray generator 3 and the first image receiver 4 operate, while the second X-ray generator 5 and the second image receiver 6 do not operate; b. The second X-ray generator 5 and the second image receiver 6 operate, while the first X-ray generator 3 and the first image receiver 4 do not operate; c. The first X-ray generator 3 and the first image receiver 4 as well as the second X-ray generator 5 and the second image receiver 6 operate simultaneously, so that the said X-ray paradoxical pulse bi-planar synchronous real-time imaging method is suitable for different operating environments.

In the embodiment, alternatively, the said operation display is used to display the operation interface of the said X-ray paradoxical pulse bi-planar synchronous real-time imaging device and also supports the parameter input for the convenience of the user to operate the said X-ray paradoxical pulse bi-planar synchronous real-time imaging device.

In the embodiment, alternatively, there is also S05 before the S10:
S05. Set the cycle top limit T of the said X-ray paradoxical pulse bi-planar synchronous real-time imaging device to determine the low limit frequency of the said X-ray paradoxical pulse bi-planar synchronous real-time imaging device, so that to further improve the synchronism of the display of the said X-ray image. Preferably, the said T is not more than 1/24 second.

In the embodiment, alternatively, there is also S35 between the S30 and S40:

S35. The said first X-ray pulse's total cycle and the cycle top limit are the same, with the duration as T₁; the said second X-ray pulse's total cycle and the cycle top limit are the same, with the duration as T₂. The overlap between the said first X-ray pulse and the said second X-ray is Tₜ, satisfying T₁+T₂≦T+Tₜ, to control the overlap amount of the pulse cycles of the said first X-ray pulse and second X-ray pulse. On the premise of increasing the energies of the said first X-ray pulse and second X-ray pulse, the interference between the said first X-ray pulse and second X-ray pulse reduces to further improve the imaging effect; preferably, the said Tₜ meets:

Tₜ≦¼×T₂.

Therefore, the interference between the said first X-ray pulse and second X-ray pulse can be further reduced.

In the embodiment, alternatively, there is also S36 between the S30 and S40: the said first X-ray pulse's total cycle and the cycle top limit are the same, with the duration as T₁; the said second X-ray pulse's total cycle and the cycle top limit are the same, with the duration as T₂, so that the said first X-ray pulse and second X-ray pulse do not mutually interfere. Preferably, the first X-ray pulse and second X-ray pulse are generated alternately.

In the embodiment, alternatively, in the said S35 or S36, the T₁ and T₂ are different, i.e., the energies of the said first X-ray pulse and second X-ray pulse are different. Then if the corresponding detection object of the first X-ray generator 3 is thicker, the T₁ can be prolonged properly and conversely, the T₁ can be reduced properly so that the examination effects of the thicker detection objects can be improved and the radiation dose of the thinner detection objects can be reduced.

In the embodiment, alternatively, the rising edge of the said second X-ray pulse is at or after the Tₜ prior to the falling edge of the said first X-ray pulse so that the overlap ratio of the said first X-ray pulse and second X-ray pulse can be controlled to improve the imaging quality. Preferably, the said Tₜ≦¼+T₂ to avoid the said second X-ray pulse is covered by the said first X-ray pulse, resulting that the image after the detection object detected by the second X-ray generator 5 can't be output in high quality.

In the embodiment, alternatively, the time zero point of the said T₁ is at the midpoint of the rising edge of the said first X-ray pulse and the ending point is at the midpoint of the falling edge of the said first X-ray pulse; the time zero point of the said T₂ is at the midpoint of the rising edge of the said second X-ray pulse and the ending point is at the midpoint of the falling edge of the said second X-ray pulse; so that the time spans of the effective energy of the said first X-ray pulse and second X-ray pulse can be calculated accurately through the said T₁ and T₂, and hence the said X-ray paradoxical pulse bi-planar synchronous real-time imaging device can be controlled more precisely.

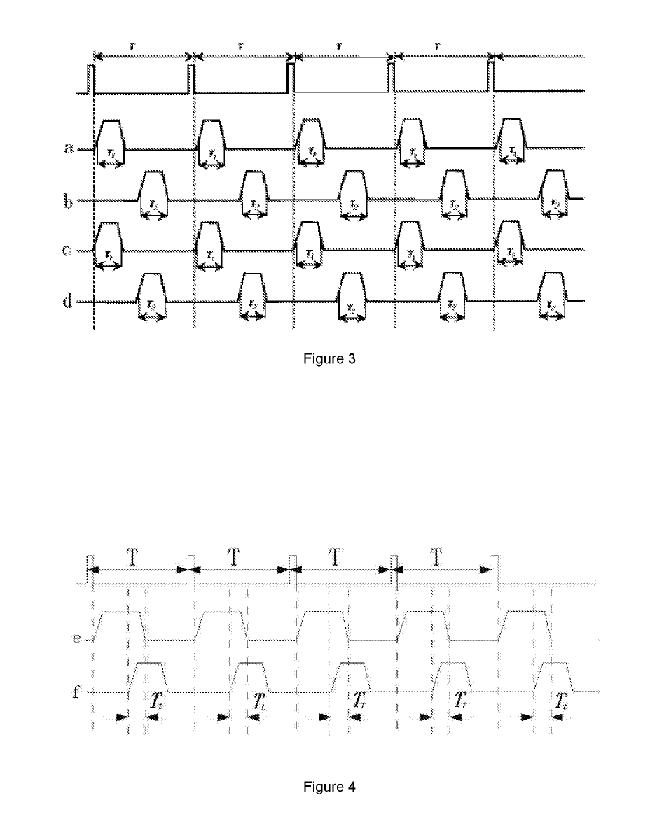

In the embodiment, the Waveform a in attached FIG. 3 is the sequence diagram of the first X-ray pulse; the Waveform b is the sequence diagram of the second X-ray pulse; the Waveform c is the sequence diagram of visible lights collected by the first camera; the Waveform d is the sequence diagram of visible lights collected by the second camera.

In the embodiment, the Waveform e in attached FIG. 3 is the sequence diagram of the first X-ray pulse; the Waveform f is the sequence diagram of the second X-ray pulse.

The sequence of the above embodiments is only for ease of description, not representing the advantages and disadvantages of the embodiments.

Finally, it shall be noted that: the above embodiments are only used to explain the technical proposals of the invention, not limit the invention; despite the invention is described in detail by reference to the above embodiments, the ordinary technical personnel in this field shall understand that: they can still modify the technical proposals recorded in all embodiments above, or replace part of the technical features with the equivalent object; but these modifications or replacements shall not constitute the nature of the corresponding technical proposals out of the spirit and range of the technical proposals in the embodiments of the invention.

1. A kind of X-ray paradoxical pulse bi-planar synchronous real-time imaging device with the features including the first C-arm, the second C-arm, support device, the first X-ray generator, the first image receiver, the second X-ray generator, the second image receiver and imaging control device;
the said first C-arm is slidably set on the said second C-arm; the said second C-arm is slidably set on the said support device;
the said first X-ray generator and the first image receiver are fixed on two axial ends of the said first C-arm respectively;
the said second X-ray generator and the second image receiver are fixed on two axial ends of the said second C-arm respectively;
taking the intersecting line between the x-ray emission plane of the said first X-ray generator and the x-ray emission plane of the said second X-ray generator as the rotation axis, the said first X-ray generator and the first image receiver rotate along the rotation axis driven by the said first C-arm and the said second X-ray generator and the second image receiver also rotate along the rotation axis driven by the said second C-arm;
the said imaging control device includes: console, image processor, X-ray controller, the first high-voltage generator, the second high-voltage generator, network card, the first camera, the second camera, the first display, the second display and operation display;
the said image processor and X-ray controller are connected to the signal of the said console;
the said first high-voltage generator and second high-voltage generator are connected to the signal of the said X-ray controller and also connected to the first X-ray generator and the second X-ray generator respectively;
the said first camera and second camera are respectively connected to the said image processor and X-ray controller by the signal of the said network card;
the said first display, second display and operation display are connected to the signal of the said image processor respectively.

2. The X-ray paradoxical pulse bi-planar synchronous real-time imaging device, according to claim 1, is characterized in that the said first X-ray generator is the first X-ray bulb tube and the said second X-ray generator is the second X-ray bulb tube.

3. The X-ray paradoxical pulse bi-planar synchronous real-time imaging device, according to claim 2, is characterized in that it also includes the first motor set on the said second C-arm, which is connected to the said console by the motor control signal.

4. The X-ray paradoxical pulse bi-planar synchronous real-time imaging device, according to claim 3, is characterized in that it also includes a foot switch, which is connected to the said console by signal.

5. The X-ray paradoxical pulse bi-planar synchronous real-time imaging device, according to claim 4, is characterized in that it includes the following steps:
S10. Control the motion of the said first motor to make the included angle between the directions of the X-ray generated from the first X-ray generator and the X-ray generated from the second X-ray generator a preset value;
S20. Select the operating mode of the said X-ray paradoxical pulse bi-planar synchronous real-time imaging device;
S30. The said first high-voltage generator and the second high-voltage generator are controlled by the said X-ray controller to generate the pulse high voltage. The said pulse high voltages generated by the said first high-voltage generator and the second high-voltage generator make the said first X-ray generator and second X-ray generator produce X-ray pulses, among which the X-ray pulse produced by the said first X-ray generator is the first X-ray pulse and the X-ray pulse produced by the said second X-ray generator is the second X-ray pulse. The phase of the said first X-ray pulse and that of the second X-ray pulse are different;
S40. The said first image receiver and second image receiver respectively receive the X-ray pulses generated when the first X-ray pulse and second X-ray pulse penetrate the examined object and generate the visible light signals;
S50. The said first camera and second camera respectively collect the visible light signals generated by the first image receiver and second image receiver and transfer to the said image processor by the network card;
S60. The said image processor displays the visible light signals collected by the said first camera on the first display and visible light signals collected by the said second camera on the second display.

6. The X-ray paradoxical pulse bi-planar synchronous real-time imaging device, according to claim 5, is characterized in that in the said S20, the said operating modes include:
a. the first X-ray generator and the first image receiver operate, while the second X-ray generator and the second image receiver do not operate;
b. the second X-ray generator and the second image receiver operate, while the first X-ray generator and the first image receiver do not operate;
c. the first X-ray generator and the first image receiver as well as the second X-ray generator and the second image receiver operate simultaneously.

7. The X-ray paradoxical pulse bi-planar synchronous real-time imaging device, according to claim 5, is characterized in that there is also S05 before the S10:
S05. Set the cycle top limit T of the X-ray paradoxical pulse bi-planar synchronous real-time imaging device.

8. The X-ray paradoxical pulse bi-planar synchronous real-time imaging device, according to claim 7, is characterized in that there is also S35 between the S30 and S40: the said first X-ray pulse's total cycle and the cycle top limit are the same, with the duration as T₁; the said second X-ray pulse's total cycle and the cycle top limit are the same, with the duration as T₂; the overlap between the said first X-ray pulse and the said second X-ray is Tt, satisfying
T₁+T₂≦T+Tₜ.

9. The X-ray paradoxical pulse bi-planar synchronous real-time imaging device, according to claim 7, is characterized in that there is also S36 between the S30 and S40: the said first X-ray pulse's total cycle and the cycle top limit are the same, with the duration as T₁; the said second X-ray pulse's total cycle and the cycle top limit are the same, with the duration as T₂, satisfying T₁+T₂≦T.

10. The X-ray paradoxical pulse bi-planar synchronous real-time imaging device, according to claim 8, is characterized in that in the said S35 or S36, the T₁ and T₂ are different.
